(19) United States
(12) Patent Application Publication
(10) Pub. No.: US 2018/0154141 A1
(43) Pub. Date: Jun. 7, 2018

(54) SKIN TREATMENT DEVICE

(71) Applicants: KOREA HEALTH INDUSTRY DEVELOPMENT INSTITUTE, Chungcheongbuk-do (KR); GOWOONSESANG COSMETICS CO., LTD, Seongnam-si, Gyeonggi-do (KR)

(72) Inventor: Gun Young AHN, Seoul (KR)

(73) Assignee: GOWOONSESANG COSMETICS CO., LTD., Seongnam-si, Gyeonggi-do (KR)

(21) Appl. No.: 15/575,082

(22) PCT Filed: Aug. 11, 2014
(86) PCT No.: PCT/KR2014/007419

§ 371 Date: Nov. 17, 2017

(30) Foreign Application Priority Data
Jun. 2, 2014 (KR) 10-2014-0067193

(51) Int. Cl. A61N 1/06; A61N 1/05; A61N 1/32

(52) U.S. Cl. CPC A61N 1/06; A61N 1/328; A61N 1/0502

(57) ABSTRACT

Disclosed is a skin treatment device including a treatment unit inserted into skin to stimulate the skin, a holder unit for detachably mounting the treatment unit thereonto to transfer a signal to the treatment unit, and a handpiece unit for mounting the holder unit thereonto to supply the signal to the holder unit, thereby rapidly exchanging the treatment unit.

### TECHNICAL FIELD

The present invention relates to a skin treatment device, and more particularly, to a skin treatment device that applies a high frequency stimulus to skin to improve a state of the skin.

### BACKGROUND ART

Generally, human skin is divided into a cornified layer, epidermis, and dermis, and the skin becomes inflamed due to blood circulation problems and incompleteness in supply of a nutrient transmitter.

Among skin inflammation, acne is a skin disease that occurs when sebum excessively producing from hair follicles is clogged with cells into the skin to cause inflammation.

In more detail, the acne occurs when cortex and corneous dead skin cells are full in hair follicles to produce yellow-white clogs, and accordingly, skin inflammation occurs around the clogs, thereby causing the clogs to protrude with red color.

If the skin inflammation is infected with bacteria, it becomes festered to yellow color, and a crater-like depression remains on the skin after a treatment for acne.

The background art of the present invention is disclosed in Korean Utility Model Registration No. 20-0335310 (dated on Nov. 27, 2003 and entitled ‘acne eraser’).

### DISCLOSURE

### Technical Problem

In a conventional practice, an insulation needle is conical, and in a process where the insulation needle is inserted into skin, accordingly, a coated portion on the insulation needle becomes rolled up by means of the skin, thereby undesirably lowering the durability of the insulation needle.

In the conventional practice, in addition, a handpiece for applying power is fixedly disposed to the insulation needle, thereby making it impossible to exchange only the insulation needle.

Accordingly, there is a need to solve the above-mentioned problems.

Accordingly, the present invention has been made in view of the above-mentioned problems occurring in the prior art, and it is an object of the present invention to provide a skin treatment device that can improve the durability of an insulation needle and exchange the insulation needle to reduce the maintenance thereof.

### Technical Solution

To accomplish the above-mentioned object, according to the present invention, there is provided a skin treatment device including: a treatment unit inserted into skin to stimulate the skin; a holder unit for detachably mounting the treatment unit thereonto to transfer a signal to the treatment unit; and a handpiece unit for mounting the holder unit thereonto to supply the signal to the holder unit.

According to the present invention, desirably, the treatment unit includes: a surgery needle inserted into the skin; a support plate connected to the surgery needle in such a manner as to be locked onto the skin to cause a limited movement thereof; and an insertion plate connected to the support plate in such a manner as to be mounted on the holder unit.

According to the present invention, desirably, the treatment unit further includes a grasping plate located between the support plate and the insertion plate in such a manner as to be graspable, the grasping plate having a shorter width than the support plate and the insertion plate.

According to the present invention, desirably, the treatment unit further includes an insulation layer applied to a portion of the support plate and a portion of the surgery needle in such a manner as to come into contact with the skin to prevent the signal from being transferred to the skin.

According to the present invention, desirably, the surgery needle is machined on an end portion thereof to form multiple surfaces thereon so that the skin becomes incised.

According to the present invention, desirably, the surgery needle is machined on an end portion thereof to form six surfaces thereon so that the skin becomes incised.

According to the present invention, desirably, the surgery needle is made of a stainless material and forms multiple surfaces on an end portion thereof by means of etching.

According to the present invention, desirably, the holder unit includes: a body part having one end portion adapted to insert the treatment unit thereinto and the other end portion adapted to be mounted on the handpiece unit so as to transfer the signal to the treatment unit; and an installation part rotatably mounted on the body part to fix the treatment unit inserted into the body part thereto.

According to the present invention, desirably, the body part includes: a holder unit body having an insertion hole adapted to insert the treatment unit thereinto and an exposed hole adapted to expose the installation part to the outside; and a signal transferring plate mounted on the holder unit body in such a manner as to come into contact with the treatment unit to transfer the signal to the treatment unit.

According to the present invention, desirably, the installation part includes: an installation button disposed on the installation hole; a locking rod extended from the installation button in such a manner as to be embedded into the holder unit body; rotary shafts disposed on the locking rod in such a manner as to be rotatably mounted on the holder unit body; a coupling protrusion protruding from the locking rod to fix the treatment unit thereto; and an elastic member embedded into the holder unit body in such a manner as to elastically support the installation button thereagainst.

### Advantageous Effects

According to the present invention, the skin treatment device has the treatment unit detachably mounted on the holder unit, thereby exchanging the treatment unit.

According to the present invention, further, the skin treatment device has the support plate locked onto the skin, thereby limiting movements of the support plate.

According to the present invention, furthermore, the skin treatment device grasps the grasping plate having a shorter width than the support plate and the insertion plate, thereby allowing the treatment unit mounted on the holder unit to be separated gently from the holder unit.

According to the present invention, in addition, the skin treatment device has the insulation layer formed on the surgery needle, thereby preventing the signal from being transferred to the surface of the skin.

According to the present invention, further, the skin treatment device has the plurality of machined surfaces formed on the surgery needle, so that if the surgery needle is inserted into the skin, the skin becomes incised to prevent the insulation layer from being damaged.

### DESCRIPTION OF DRAWINGS

FIG. 1 is a schematic perspective view showing a skin treatment device according to the present invention.

FIG. 2 is a schematic side view showing a treatment unit of the skin treatment device according to the present invention.

FIG. 3 is a schematic side view showing the state where the treatment unit of FIG. 2 is inserted into skin.

FIG. 4 is a schematic perspective view showing a holder unit of the skin treatment device according to the present invention.

FIG. 5 is a schematic sectional view showing the state where the holder unit and the treatment unit are coupled to each other in the skin treatment device according to the present invention.

FIG. 6 is a schematic sectional view showing the state where the holder unit and the treatment unit are released from their coupled state in the skin treatment device according to the present invention.

FIG. 7 is a schematic perspective view showing a body part of the skin treatment device according to the present invention.

FIG. 8 is a schematic sectional view showing an installation part of the skin treatment device according to the present invention.

### MODE FOR INVENTION

Hereinafter, an explanation on a skin treatment device according to the present invention will be in detail given with reference to the attached drawing. In the description, the thicknesses of the lines or the sizes of the components shown in the drawing may be magnified for the clarity and convenience of the description. Further, the terms as will be discussed later are defined in accordance with the functions of the present invention, but may be varied under the intention or regulation of a user or operator. Therefore, they should be defined on the basis of the whole scope of the present invention.

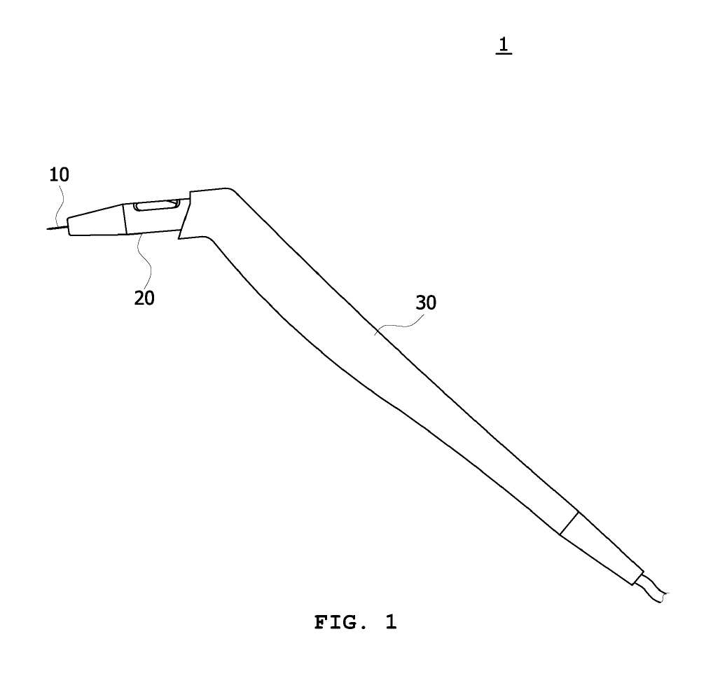

FIG. 1 is a schematic perspective view showing a skin treatment device according to the present invention. Referring to FIG. 1, a skin treatment device 1 according to the present invention includes a treatment unit 10, a holder unit 20, and a handpiece unit 30. The skin treatment device 1 applies a signal, that is, a high frequency current to skin to remove inflammation from the skin or to improve the conditions of the skin.

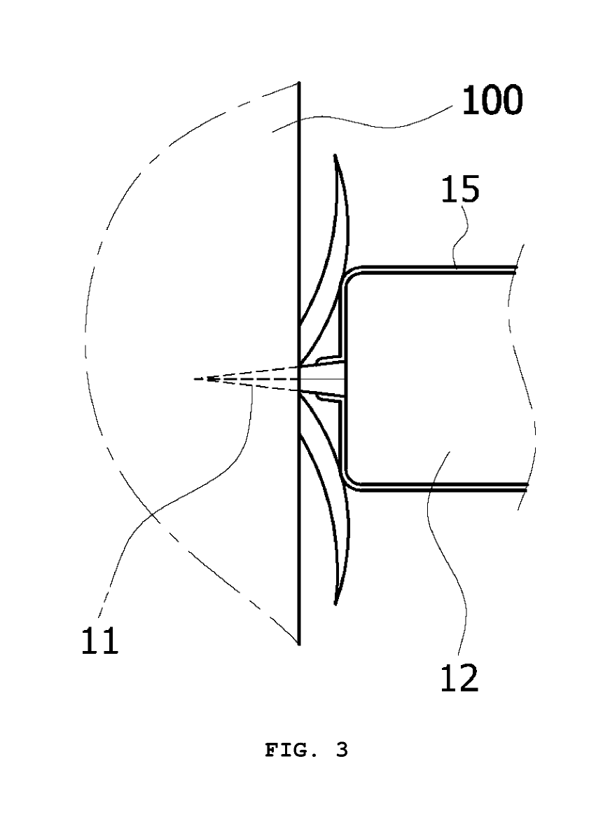

The treatment unit 10 is inserted into a patient's skin 100 to stimulate the skin 100 (See FIG. 3), and the holder unit 20 is coupled to the treatment unit 10 to transfer a signal to the treatment unit 10. At this time, the treatment unit 10 is detachably attached to the holder unit 20. The handpiece unit 30 is coupled to the holder unit 20 to supply the signal to the holder unit 20. The handpiece unit 30 is provided in a given shape so as to be graspable by a user′ hand and includes a signal supply part (not shown) embedded therein in such a manner as to be connected to the holder unit 20 to supply the signal to the holder unit 20. On the other hand, the skin treatment device 1 according to the present invention further includes an earth unit (not shown) coming into contact with the patient to constitute a circuit with his or her body and a switch unit (not shown) for applying power to the handpiece unit 30.

That is, human skin tissue is divided into epidermis and subcutaneous tissue located on underside of the epidermis. The epidermis having a cornified layer acts as a biological barrier against an environment, and melanocytes are located in the bottom layer of the epidermis. In addition, the subcutaneous tissue on the underside of the epidermis has a collagen protein as a support to maintain the structure of the skin. The collagen is a protein that is synthesized by fibroblast and has a helical structure of three poly peptides. If a human body becomes injured, the human body starts to perform a complicate repairing function, and in this case, the fibroblast, which exists under the skin, becomes activated and temporarily enters ECM (Extra Cellular Matrix) around the injury to produce collagen that covers the injury as soon as possible. In a process where the fibroblast slowly moves around the matrix and pulls fibers to combine the fibers again, the fibroblast makes the matrix gradually hardened, and because of growth elements contained in the matrix being soft in an initial state, the fibroblast reveals a contractile protein through the growth elements, so that if it reaches a given time point, it stops and changes to a strong contractile cells like Popeye and stays in the matrix to pull the edge of the injury, thereby providing a scar or making the surface of the skin irregular. At this time, the collagen fibrous tissue is heated by a given energy source, and the physical properties of the protein matrix become deformed at a specific temperature. Accordingly, the molecular bonding of the matric becomes broken to permit the state of the skin to be evenly made. Also, the collagen layer is activated to return the elasticity of the skin to an original state.

Using the above-mentioned principle, high frequency energy is irradiated to thus reach the subcutaneous tissue on the underside of the epidermis, and a temperature of the subcutaneous tissue is raised, thereby achieving the remodelling of the skin tissue. Especially, because high frequency current has a substantially short width of vibration when applied to a human body tissue, a motion of ions is not almost caused, and also, the high frequency current does not cause any electrochemical reaction or electrolysis. If high frequency electric energy is applied to a tissue, molecules constituting the tissue become vibrated and frictional with each other whenever the directions of currents are changed, thereby generating bioheat. Further, the high frequency current does not stimulate any sensory nerves or motor nerves and also does not generate any uncomfortability or muscle contraction in the human body, unlike other currents, and advantageously generates heat energy. The heat energy stimulates the functions of cells and increases the amount of blood flow. In the treatment of department of dermatology, the application of the high frequency current is widely used for a variety of fields such as acne treatment, lifting, acne scar treatment, filler injection, and the like.

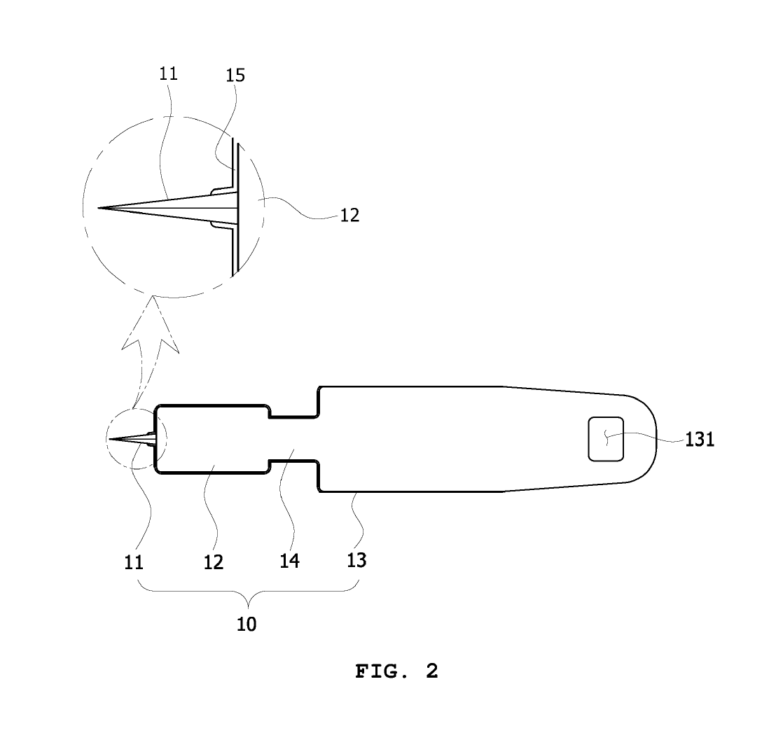

FIG. 2 is a schematic side view showing a treatment unit of the skin treatment device according to the present invention, and FIG. 3 is a schematic side view showing the state where the treatment unit of FIG. 2 is inserted into skin. Referring to FIGS. 2 and 3, the treatment unit 10 of the skin treatment device 1 according to the present invention includes a surgery needle 11, a support plate 12, and an insertion plate 13. The treatment unit 10 is made of a conductive material so that it can transfer the signal to the skin 100.

The surgery needle 11 is inserted into the skin 100. The surgery needle 11 is made of a conductive material so that the signal transferred from the holder unit 20 can be supplied to an interior of the skin 100.

The support plate 12 has a shape of a thin plate in such a manner as to be connected to the surgery needle 11. The support plate 12 has a longer width than the surgery needle 11, so that when the surgery needle 11 is inserted into the skin 100, the support plate 12 is locked onto the skin 100 to cause limited movement. For example, the left end of the surgery needle 11 is first inserted into the skin 100 (See FIG. 3), and the support plate 12 has a larger sectional area than the surgery needle 11, so that in the process where the surgery needle 11 is inserted into the skin 100, the left end of the support plate 12 comes into contact with the surface of the skin 100 to limit an inserted depth of the surgery needle 11.

The insertion plate 13 is connected to the support plate 12 and is thus mounted on the holder unit 20. For example, the insertion plate 13 is provided in a shape of a thin plate and has a needle hole 131 adapted to be stably coupled to the holder unit 20. Accordingly, the surgery needle 11, the support plate 12, and the insertion plate 13 are sequentially arranged from the left side of the treatment unit 10.

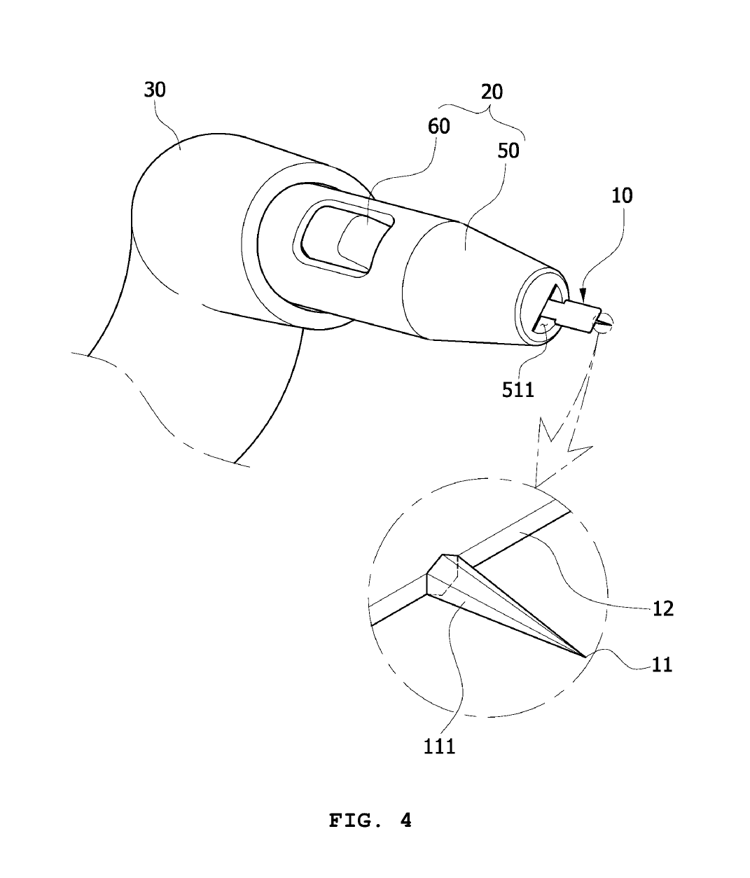

According to the present invention, the treatment unit 10 further includes a grasping plate 14. The grasping plate 14 is located between the support plate 12 and the insertion plate 13 in such a manner as to have both ends connected to the support plate 12 and the insertion plate 13. That is, the surgery needle 11, the support plate 12, the grasping plate 14, and the insertion plate 13 are sequentially arranged from the left side of the treatment unit 10. The grasping plate 14 has a shorter width than the support plate 12 and the insertion plate 13. When the insertion plate 13 is mounted on the holder unit 20, further, the grasping plate 14 is exposed to the outside (See FIG. 4). While the treatment unit 10 is being moved by holding an edge of the grasping plate 14 with a tool like tweezers, even if the tool slides on the grasping plate 14, it is locked onto the support plate 12 or the insertion plate 13, thereby ensuring the movabilty of the treatment unit 10.

According to the present invention, the treatment unit 10 further includes an insulation layer 15. The insulation layer 15 is applied to a portion of the support plate 12 and a portion of the surgery needle 11 to prevent the signal from being transferred to the surface of the skin 100. If the insulation layer 15 is formed on the left end portion of the support plate 12 and on the right end portion of the surgery needle 11 when viewed in FIG. 3, the surface of the skin 100 comes into contact with the insulation layer 15 when the surgery needle 11 is inserted into the skin 100, thereby preventing the signal from being transferred to the surface of the skin 100.

In addition to the portion of the support plate 12 and the portion of the surgery needle 11, the insulation layer 15 may be applied evenly to the support plate 12 and the grasping plate 14, thereby preventing the occurrence of electric shocks in the process where the treatment unit 10 is grasped by the user. On the other hand, the insertion plate 13 is electrically connected to the holder unit 20, and accordingly, the insulation layer 15 is not formed on the insertion plate 13. Otherwise, the insulation layer 15 is formed only on a portion of the insertion plate 13 so as to be electrically connectable to the holder unit 20.

FIG. 4 is a schematic perspective view showing the holder unit of the skin treatment device according to the present invention. Referring to FIGS. 2 to 4, the surgery needle 11 of the treatment unit 10 according to the present invention is machined on an end portion thereof to form multiple surfaces thereon so that it can incise the skin 100.

If the end portion of the surgery needle 11 has a shape of a cone, that is, the skin is not incised when the end portion of the surgery needle 11 is piercedly inserted into the skin 100. When the surgery needle 11 is completely inserted into the skin 100, accordingly, the skin 100 pushes the insulation layer 15 to cause the durability of the insulation layer 15 coated on the surgery needle 11 to be deteriorated. According to the present invention, however, the surgery needle 11 has machined surfaces 111 so that when it is inserted into the skin 100, the skin 100 can be incised. Even if the surgery needle 11 is completely inserted into the skin 100, accordingly, the skin 100 does not push the insulation layer 15 to improve the durability of the insulation layer 15 coated on the surgery needle 11.

In more detail, the surgery needle 11 has six machined surfaces 111 so as to incise the skin 100. The machined surfaces 111 are formed on the outer periphery of the surgery needle 11 and have lengths along the axial direction of the surgery needle 11. At this time, the surgery needle 11 becomes reduced in a sectional area as it goes toward the end portion thereof. If the machined surfaces 111 are formed on the end portion of the surgery needle 11, the end portion of the surgery needle 11 is piercedly inserted into the surface of the skin 100, so that the surface of the skin 100 is incised to open (See FIG. 3).

The surgery needle 11 is made of a stainless material among metal materials having conductivity. At this time, the surgery needle 11 has the machined surfaces 111 formed through etching.

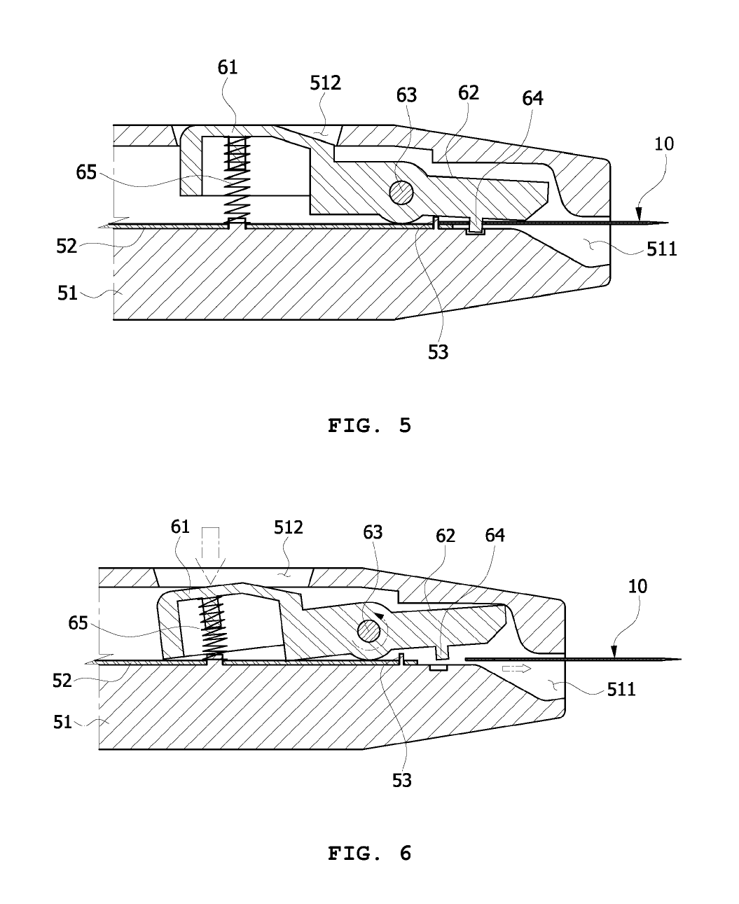

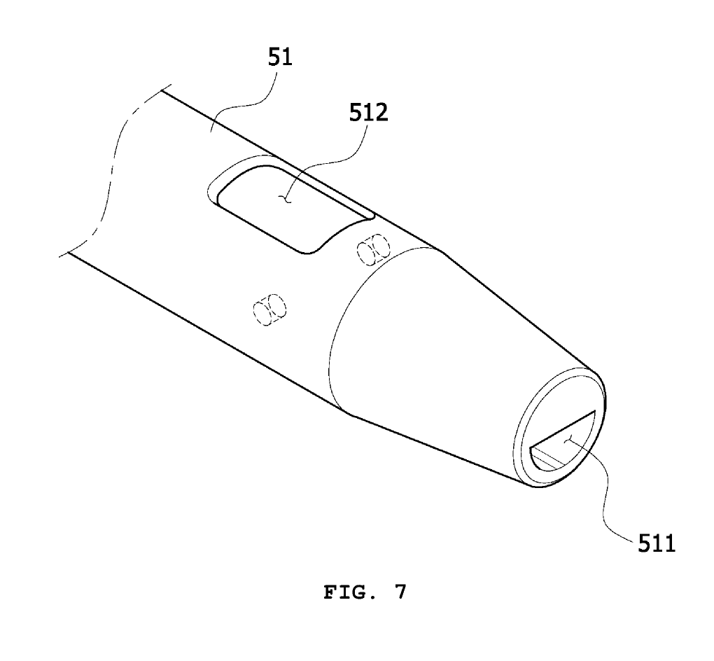

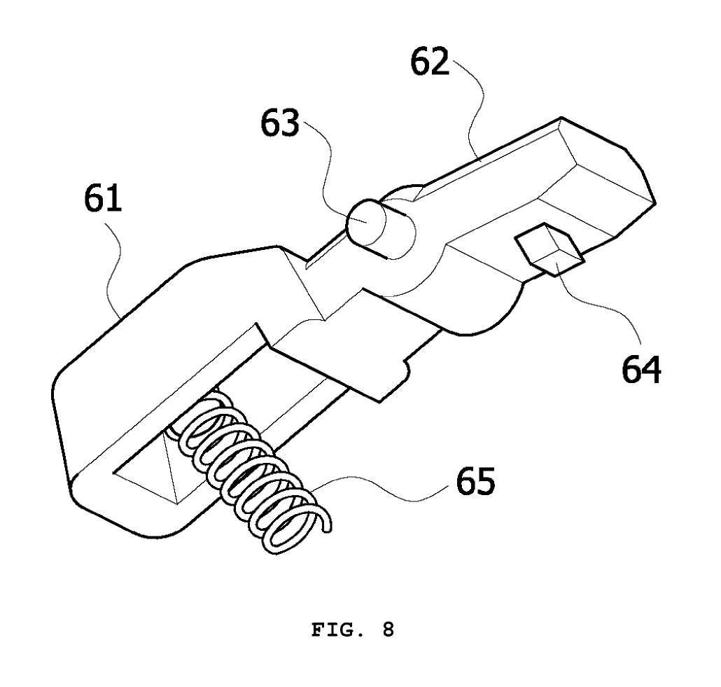

FIG. 5 is a schematic sectional view showing the state where the holder unit and the treatment unit are coupled to each other in the skin treatment device according to the present invention, FIG. 6 is a schematic sectional view showing the state where the holder unit and the treatment unit are released from their coupled state in the skin treatment device according to the present invention, FIG. 7 is a schematic perspective view showing a body part of the skin treatment device according to the present invention, and FIG. 8 is a schematic sectional view showing an installation part of the skin treatment device according to the present invention. Referring to FIGS. 4 to 8, the holder unit 20 of the skin treatment device 1 according to the present invention includes a body part 50 and an installation part 60.

The body part 50 has one end portion adapted to insert the treatment unit 10 thereinto and the other end portion adapted to be mounted on the handpiece unit 30. The installation part 60 is rotatably mounted on the body part 50 to fix the treatment unit inserted into the body part 50 thereto. For example, the installation part 60 is coupled to the insertion plate 13 to fix the insertion plate 13 thereto.

According to the present invention, the body part 50 includes a holder unit body 51 and a signal transferring plate 52.

The holder unit body 51 has an insertion hole 511 formed on one end portion thereof and an exposed hole 512 formed on a center portion thereof. The insertion hole 511 forms a space into which the treatment unit 10 is inserted, and the exposed hole 512 exposes the installation part 60 embedded into the holder unit body 51 to the outside. The holder unit body 51 may have a pair of bodies symmetrical to each other.

The signal transferring plate 52 is mounted on the holder unit body 51. The signal transferring plate 52 is made of a conductive material and comes into contact with the treatment unit 10 to transfer the signal to the treatment unit 10. For example, the signal transferring plate 52 is embedded into the holder unit body 51 in such a manner as to allow an end portion thereof to come into contact with the signal supply part (not shown) of the handpiece unit 30. Also, the insertion plate 13 inserted into the holder unit body 51 is brought into contact with the signal transferring plate 52.

The holder unit body 51 has a stopper 53. The stopper 53 protrudes from the holder unit body 51 to lock the insertion plate 13 inserted into the insertion hole 511 thereonto so that an inserted distance of the insertion plate 13 can be limited.

According to the present invention, the installation part 60 includes an installation button 61, a locking rod 62, rotary shafts 63, a coupling protrusion 64, and an elastic member 65.

The installation button 61 is embedded into the holder unit body 51 in such a manner as to be exposed to the outside through the installation hole 512.

The locking rod 62 is extended from the installation button 61. The locking rod 62 is embedded into the holder unit body 51.

The rotary shafts 63 are disposed on the locking rod 62 in such a manner as to be rotatably mounted on the holder unit body 51. For example, the rotary shafts 63 protrude from both sides of the locking rod 62 in such a manner as to be coupled to the holder unit body 51 by means of pins.

The coupling protrusion 64 protrudes downwardly from underside of the locking rod 62 to fix the treatment unit 10 thereto. For example, the coupling protrusion 64 passes through the needle hole 131 to limit movement of the insertion plate 13.

The elastic member 65 is embedded into the holder unit body 51 to elastically support the installation button 61 thereagainst. For example, the installation button 61 has a shape of a cap having an open lower portion, and the elastic member 65 is inserted into the installation button 61.

At this time, the elastic member 65 has a shape of a coil spring for winding protrusions formed on the installation button and the holder unit body 51, thereby suppressing left and right movements of the installation button 61 and the holder unit body 51.

The coupling protrusion 64 is formed on a right side around the rotary shafts 63, and the elastic member 65 is located on a left side around the rotary shafts 63 (See FIGS. 5 and 6).

If the rotary shafts 63 rotate in a clockwise direction by means of a restoring force of the elastic member 65, accordingly, the coupling protrusion 64 moves downward to fix the insertion plate 13 thereto.

If the elastic member 65 is contracted by means of the pressurization of the installation button 61 by the user, further, the rotary shafts 63 rotate in a counterclockwise direction, so that the coupling protrusion 64 moves upward to allow the insertion plate 13 to be released from the fixed state thereto.

Under the above-mentioned configuration, now, an explanation on the operation and effect of the skin treatment device according to the present invention will be given.

If the holder unit 20 is mounted on the handpiece unit 30, the signal transferring plate 52 embedded into the holder unit 20 is kept connected to the signal supply part (not shown) of the handpiece unit 30.

In this state, if the installation button 61 is pressurized by the user, the elastic member 65 is contracted, and the coupling protrusion 64 moves upward (See FIG. 6).

If the installation button 61 is pressurized, the grasping plate 14 is held by means of a tool to insert the insertion plate 13 into the insertion hole 511. At this time, the insertion plate 13 is inserted until it is locked onto the stopper 53.

If the insertion of the insertion plate 13 is finished, an external force applied to the installation button 61 is removed. If the external force applied to the installation button 61 is removed, the coupling protrusion 64 moves downward by means of the restoring force of the elastic member 65 and is thus located on the needle hole 131 (See FIG. 5).

If the treatment unit 10 is mounted on the holder unit 20, it is inserted into the skin 100 by the user (See FIG. 3). At this time, the machined surfaces 111 are formed on the surgery needle 11 located on the end portion of the treatment unit 10, so that they incise the skin 100 to keep the insulation layer 15 from being damaged due to the pressure of the skin 100.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

What is claimed is:

1. A skin treatment device comprising:
a treatment unit inserted into skin to stimulate the skin;
a holder unit for detachably mounting the treatment unit thereonto to transfer a signal to the treatment unit; and
a handpiece unit for mounting the holder unit thereonto to supply the signal to the holder unit.

2. The skin treatment device according to claim 1, wherein the treatment unit comprises:
a surgery needle inserted into the skin;
a support plate connected to the surgery needle in such a manner as to be locked onto the skin to cause a limited movement thereof; and
an insertion plate connected to the support plate in such a manner as to be mounted on the holder unit.

3. The skin treatment device according to claim 2, wherein the treatment unit further comprises a grasping plate located between the support plate and the insertion plate in such a manner as to be graspable, the grasping plate having a shorter width than the support plate and the insertion plate.

4. The skin treatment device according to claim 2, wherein the treatment unit further comprises an insulation layer applied to a portion of the support plate and a portion of the surgery needle in such a manner as to come into contact with the skin to prevent the signal from being transferred to the skin.

5. The skin treatment device according to claim 2, wherein the surgery needle is machined on an end portion thereof to form multiple surfaces thereon so that the skin becomes incised.

6. The skin treatment device according to claim 2, wherein the surgery needle is machined on an end portion thereof to form six surfaces thereon so that the skin becomes incised.

7. The skin treatment device according to claim 2, wherein the surgery needle is made of a stainless material and forms multiple surfaces on an end portion thereof by means of etching.

8. The skin treatment device according to claim 1, wherein the holder unit comprises:
a body part having one end portion adapted to insert the treatment unit thereinto and the other end portion adapted to be mounted on the handpiece unit so as to transfer the signal to the treatment unit; and
an installation part rotatably mounted on the body part to fix the treatment unit inserted into the body part thereto.

9. The skin treatment device according to claim 8, wherein the body part comprises:
a holder unit body having an insertion hole adapted to insert the treatment unit thereinto and an exposed hole adapted to expose the installation part to the outside; and
a signal transferring plate mounted on the holder unit body in such a manner as to come into contact with the treatment unit to transfer the signal to the treatment unit.

10. The skin treatment device according to claim 9, wherein the installation part comprises:
an installation button disposed on the installation hole;
a locking rod extended from the installation button in such a manner as to be embedded into the holder unit body;
rotary shafts disposed on the locking rod in such a manner as to be rotatably mounted on the holder unit body;
a coupling protrusion protruding from the locking rod to fix the treatment unit thereto; and
an elastic member embedded into the holder unit body in such a manner as to elastically support the installation button thereagainst.
